(19) United States
(12) Patent Application Publication
(10) Pub. No.: US 2016/0090637 A1
(43) Pub. Date: Mar. 31, 2016

(54) Pharmacogenomic Biomarkers for B Cell Malignancies and Methods of Use Thereof

(71) Applicant: Wei CHEN, Edina, MN (US)

(72) Inventor: Wei Chen, Edina, MN (US)

(21) Appl. No.: 14/891,184

(22) PCT Filed: May 13, 2014
(86) PCT No.: PCT/US14/37848

§ 371 Date: Nov. 13, 2015

Related U.S. Application Data
(60) Provisional application No. 61/823,246, filed on May 14, 2013.

(51) Int. Cl. C12Q 1/68; A61K 38/20

(52) U.S. Cl. CPC C12Q 1/6886; A61K 38/2066; C12Q 2600/106; C12Q 2600/156

(57) ABSTRACT

The present invention relates to diagnosing drug-resistant chronic lymphocytic leukemia (CLL) in a mammal.

### BACKGROUND OF THE INVENTION

Interleukin 10 (IL-10) is an anti-inflammatory cytokine produced by cells of body immune system (e.g., monocytes, Tregs). It has potent effects in suppression of T cell and NK cell responses, the regulation of survival and function of B cells, and the regulation of JAK-STAT signaling pathway. IL-10 interacts with its specific receptor IL-10R1 (also called IL-10 receptor alpha subunit) but also requires IL-10R2 subunit to initiate signaling. IL10RA is the gene that encodes the IL-10R1 protein.

Treatments are currently available for CLL patients, but there are some patients that respond to the current treatments, and some that do not (i.e., those that are drug-resistant). There is a need for diagnostics to determine prior to treatment whether a patient will likely be successfully treated with the current therapies, or if they are drug-resistant.

### SUMMARY OF THE INVENTION

The present invention relates to the discovery that deletion and mutation of the IL10RA gene on chromosome 11 q23 avert CpG ODN drug-induced apoptosis of human B-CLL cells. The IL10RA gene encodes a protein IL-10R1 precursor, where the IL-10R1 precursor contains a 21 amino acid signal peptide at the N-terminal. IL-10R1 precursor turns into IL-10R1 by posttranslational modification after the 21 amino acid signal peptide is cleaved off. These new biomarkers are useful as pharmacogenomic biomarker classifiers. These biomarkers can be used to select, stratify, or exclude patients with B cell malignancies for certain therapies and can be used to predict clinical outcomes. These biomarkers and diagnostic methods also can be applied to all human B cell malignancies, in addition to CLL, including all types of B cell leukemias, lymphomas, and multiple myelomas, and more. The present invention provides a method for detecting the presence of a biomarker associated with drug-resistant B cell malignancy. In certain embodiments, the drug resistance is CpG-B ODN drug resistance. In certain embodiments, the B cell malignancy is drug-resistant chronic lymphocytic leukemia (CLL).

In one embodiment of the invention, the method involves obtaining a physiological sample from a mammal, wherein the sample comprises nucleic acid, and determining the presence of the biomarker. As used herein, the phrase “physiological sample” is meant to refer to a biological sample obtained from a mammal that contains nucleic acid. For example, a physiological sample can be a sample collected from an individual, where the physiological sample contains chronic lymphocytic leukemia (B-CLL) cells. Such samples include, but not limited to, e.g., a cell sample, such as a blood cell, e.g., a lymphocyte, a peripheral blood cell; a sample collected from the spinal cord; a tissue sample such as cardiac tissue or muscle tissue, e.g., cardiac or skeletal muscle; an organ sample, e.g., liver or skin; a hair sample, e.g., a hair sample with roots; and/or a fluid sample, such as blood. In certain embodiments, the sample is peripheral blood, umbilical cord blood, lymph nodes, bone marrow, amniotic fluid, or tumor specimens. In certain embodiments the test for IL10RA can be in any cell, tissue, hair, body fluid samples that contain DNA.

The term “biomarker” is generally defined herein as a biological indicator, such as a particular molecular feature, that may affect or be related to diagnosing or predicting an individual's health. For example, in certain embodiments of the present invention, the biomarker comprises a mutant IL10RA gene, such as an allele of IL10RA that has a substitution of A to G at nucleotide 552 of the precursor gene (wildtype is provided as SEQ ID NO:2 in FIG. 7B). This corresponds to nucleotide 536 of the mature IL-10R1. The amino acid sequence for IL-10R1 is provided as SEQ ID NO:1 (the 21 amino acids of the signal sequence are indicated by italics in FIG. 7A). This mutation encodes an IL-10R1 having the serine at position 159 of the precursor protein that is replaced with a Glycine (i.e., S159G mutation). In other embodiments, the serine at position 159 is deleted. In other embodiments, a region of the IL-10R1 comprising the amino acid at position 159 is deleted. It has been discovered that even a single amino acid point mutation or deletion in IL-10R1 (e.g., S138G of the mature IL-10R1 or S159G of the IL-10R1 precursor) affects IL-10 binding or IL-10 signaling. In certain embodiments, a single base is detected, whereas in other embodiments, a longer deletion that includes part or the whole sequence encoding the IL-10R1 or the IL-10R1 precursor is detected.

“Oligonucleotide probe” can refer to a nucleic acid segment, such as a primer, that is useful to amplify a sequence in the IL10RA gene that is complementary to, and hybridizes specifically to, a particular sequence in IL10RA. Genomic DNA extracted from a sample can be detected for IL-10R1 mutation using specific primers and by bidirectional polymerase chain reaction amplification of specific alleles.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

In one embodiment of the present invention, the method involves contacting the sample with at least one oligonucleotide probe to form a hybridized nucleic acid and amplifying the hybridized nucleic acid. “Amplifying” utilizes methods such as the polymerase chain reaction (PCR), ligation amplification (or ligase chain reaction, LCR), strand displacement amplification, nucleic acid sequence-based amplification, and amplification methods based on the use of Q-beta replicase. These methods are well known and widely practiced in the art. Reagents and hardware for conducting PCR are commercially available. For example, in certain embodiments of the present invention, IL10RA, or a portion thereof, may be amplified by PCR. In another embodiment of the present invention, at least one oligonucleotide probe is immobilized on a solid surface.

In certain embodiments, the present invention provides a method for detecting the presence of a biomarker in a mammal, comprising identifying in a nucleic acid sample from the mammal a guanine (G) at nucleotide 552 of SEQ ID NO:2 or a deletion of an adenosine (A) at nucleotide 552 of SEQ ID NO:2. In certain embodiments, the mammal is a human that has a B cell malignancy, such as chronic lymphocytic leukemia (CLL). In certain embodiments, the method comprises identifying in a nucleic acid sample from the mammal a guanine (G) at nucleotide 552. In certain embodiments, the method comprises identifying in a nucleic acid sample from the mammal a deletion of an adenosine (A) at nucleotide 552 of SEQ ID NO:2. In certain embodiments, the mammal is homozygous for the biomarker.

In certain embodiments, the present invention provides a method for detecting the presence of a biomarker in a mammal, comprising identifying in an amino acid sample from the mammal a glycine (G) at residue 159 of SEQ ID NO:1 or a deletion of a serine (S) at residue 159 of SEQ ID NO:1. In certain embodiments, the mammal is a human that has a B cell malignancy, such as chronic lymphocytic leukemia (CLL). In certain embodiments, the method comprises identifying in an amino acid sample from the mammal a glycine (G) at residue 159 of SEQ ID NO:1. In certain embodiments, the method comprises identifying in a nucleic acid sample from the mammal a deletion of serine (S) at residue 159 of SEQ ID NO:1. In certain embodiments, the mammal is homozygous for the biomarker.

In certain embodiments, the present invention provides a method for detecting the presence of drug-resistant a B cell malignancy, such as chronic lymphocytic leukemia (CLL) in a mammal, comprising identifying a biomarker in a nucleic acid sample from the mammal nucleotide 552 of SEQ ID NO:2, wherein the presence of a guanine (G) at nucleotide 552 or the deletion of an adenosine (A) at nucleotide 552 in both alleles is indicative of the mammal having drug-resistant chronic lymphocytic leukemia. In certain embodiments, the method further comprises contacting the sample with at least one oligonucleotide probe to form a hybridized nucleic acid and amplifying the hybridized nucleic acid. In certain embodiments, IL10RA or a portion thereof is amplified. In certain embodiments, the amplification of the hybridized nucleic acid is carried out by polymerase chain reaction, strand displacement amplification, ligase chain reaction, or nucleic acid sequence-based amplification. In certain embodiments, the at least one oligonucleotide probe is immobilized on a solid surface. In certain embodiments, the drug resistance is CpG-A oligodeoxynucleotide (ODN), CpG-B ODN, or CpG-C ODN drug resistance. In certain embodiments, the drug resistance is CpG-B ODN drug resistance. At least three distinct classes of CpG ODN with structural and functional differences have been identified. [Krieg A M. Development of TLR9 agonists for cancer therapy. J Clin Invest. 2007; 117(5):1184-1194; Vollmer, J., R. Weeratna, P. Payette, M. Jurk, C. Schetter, M. Laucht, T. Wader, S. Tluk, M. Liu, H. L. Davis, and A. M. Krieg. 2004. Characterization of three CpG oligodeoxynucleotide classes with distinct immunostimulatory activities. Eur J Immunol 34:251-262.] CpG-B ODNs are most potent TLR9 agonists stimulating normal B cells and induce apoptosis of leukemic B cells. [Hartmann, G., and A. M. Krieg. 2000. Mechanism and function of a newly identified CpG DNA motif in human primary B cells. J Immunol 164:944; Liang X, Moseman E A, Farrar M A, et al. Toll-like receptor 9 signaling by CpG-B oligodeoxynucleotides induces an apoptotic pathway in human chronic lymphocytic leukemia B cells. Blood; 115(24):5041-5052.]

In certain embodiments, the present invention provides a method for detecting the presence of an drug-resistant chronic lymphocytic leukemia in a mammal, comprising identifying in a nucleic acid sample from the mammal nucleotide 552 of SEQ ID NO:2, wherein the presence of a guanine (G) nucleotide at nucleotide 552 or the deletion of an adenosine (A) at nucleotide 552 in both alleles is indicative of the mammal being having drug-resistant chronic lymphocytic leukemia. In certain embodiments, the method further comprises contacting the sample with at least one oligonucleotide probe to form a hybridized nucleic acid and amplifying the hybridized nucleic acid. In certain embodiments, IL10RA or a portion thereof is amplified. In certain embodiments, the amplification of the hybridized nucleic acid is carried out by polymerase chain reaction, strand displacement amplification, ligase chain reaction, or nucleic acid sequence-based amplification. In certain embodiments, at least one oligonucleotide probe is immobilized on a solid surface.

In certain embodiments, the present invention provides a method for detecting the presence of a biomarker associated with drug-resistant a B cell malignancy, such as drug-resistant chronic lymphocytic leukemia (CLL), comprising determining the presence of the biomarker in a physiological sample from a mammal, wherein the sample comprises nucleic acid. In certain embodiments, the method further comprises contacting the sample with at least one oligonucleotide probe to form a hybridized nucleic acid and amplifying the hybridized nucleic acid. In certain embodiments, IL10RA or a portion thereof is amplified. In certain embodiments, the amplification of the hybridized DNA is carried out by polymerase chain reaction, strand displacement amplification, ligase chain reaction, or nucleic acid sequence-based amplification. In certain embodiments, at least one oligonucleotide probe is immobilized on a solid surface. In certain embodiments, the biomarker comprises an IL10RA gene having a Guanine (G) at nucleotide 552 of SEQ ID NO:2 or the deletion of an adenosine (A) at nucleotide 552 of SEQ ID NO:2. In certain embodiments, IL10RA gene encodes a protein having a Glycine (G) at amino acid residue 159 of SEQ ID NO:1.

In certain embodiments, the present invention provides a method for diagnosing drug-resistant a B cell malignancy, such as drug-resistant chronic lymphocytic leukemia (CLL) in a mammal comprising detecting the presence of a biomarker in a physiological sample from the mammal the sample, wherein the presence of the biomarker is indicative of drug-resistant CLL. In certain embodiments, the sample comprises nucleic acid. In certain embodiments, the method further comprises contacting the sample with at least one oligonucleotide probe to form a hybridized nucleic acid and amplifying the hybridized nucleic acid. In certain embodiments, IL10RA or a portion thereof is amplified. In certain embodiments, the amplification of the hybridized DNA is carried out by polymerase chain reaction, strand displacement amplification, ligase chain reaction, or nucleic acid sequence-based amplification. In certain embodiments, the biomarker comprises an IL10RA gene has a Guanine at nucleotide 552 of SEQ ID NO:2 or a deletion of an adenosine (A) at nucleotide 552 of SEQ ID NO:2. In certain embodiments, the method of claim 34, wherein the IL10RA gene encodes a protein that has a Glycine (G) at amino acid residue 159 of SEQ ID NO:1. In certain embodiments, the biomarker is a IL-10R1 protein. In certain embodiments, the IL-10R1 protein has a Glycine (G) at amino acid residue 159 of SEQ ID NO:1.

Further provided by the present invention is a kit comprising a diagnostic test for detecting the presence of drug-resistant a B cell malignancy, such as CLL in a mammal comprising packaging material, containing, separately packaged, at least one oligonucleotide probe capable of forming a hybridized nucleic acid with IL10RA and instructions means directing the use of the probe in accord with the methods of the invention.

In certain embodiments, the present invention further provides administering IL-10 for treatment human B cell malignancies. Our finding that IL-10RA gene deletion and mutation affect IL-10 induced apoptosis of CLL, which is IL-10 treatment time and drug dose dependent. This also needs to be protected for IL-10 as a drug, not just for CpG ODN drugs.

### BRIEF DESCRIPTION OF DRAWINGS

FIGS. 1A-1D. B-CLL cells from a subset of CLL patients are resistant to CpG-B ODN or IL-10 induced apoptosis. (A) Purified B-CLL cells were cultured with or without CpG-B ODN or IL-10. Data are viable B-CLL cell counts at day 5 cultures of 15 CpG-sensitive B-CLL samples (left panel) and 11 CpG-resistant samples (right panel) from CLL patients. Each dot represents an individual patient and the horizontal bar represents the median level. *P<0.01, comparing B-CLL cell numbers in cultures with or without CpG-B ODN or IL-10. (B) Representative results of TMRE or Annexin V/PI staining performed to determine the number of viable and apoptotic B-CLL cells at day 5 cultures with or without CpG-B ODN or IL-10. (C) Cleavage of caspase-9, caspase-3, and PARP in B-CLL cells cultured with or without CpG-B ODN or IL-10 were determined by western blot at day 5 cultures. Data are results of 3 independent experiments with B-CLL cells from 3 CpG-sensitive and 3 CpG-resistant CLL samples. (D) Representative results of intracellular staining of cleaved caspase-3 (shaded histogram) in B-CLL cells at day 5 cultures with or without CpG-B ODN or IL-10, indicated with MFI number, and overlaid with isotype control (unshaded histogram).

FIGS. 2A-2B. IL-10 induces apoptosis of CpG-sensitive but not CpG-resistant B-CLL cells in a treatment time- and dose-dependent manner. (A) B-CLL cells were cultured with various doses of IL-10 for 5 days. Kinetic changes of the number (%) of viable B-CLL cells in cultures with the indicated IL-10 doses at day 5 cultures were determined. (B) B-CLL cells were cultured with or without IL-10 (10 ng/ml) for 9 days. Kinetic changes of the number (%) of viable B-CLL cells in cultures were determined at each indicated time point. Data are representative results from 3 independent experiments with B-CLL cells from 3 IL-10 sensitive (left panels) and 3 IL-10 resistant (right panels) CLL patients.

FIGS. 3A-3B. Both CpG-B ODN and IL-10 induce apoptosis of CpG-sensitive but not CpG-resistant B-CLL cells via the mitochondrial apoptotic pathway. (A) Western blot of Bax translocation to mitochondria [M] and cytochrome c release to cytosol [C] in B-CLL cells from CpG-sensitive and CpG-resistant samples at day 3 cultures with or without CpG-B ODNs or IL-10. The bar diagram shows the percentage of Bax or cytochrome c level in cytosol or mitochondria to the total Bax or cytochrome c level in B-CLL cells. Densitometry values of Bax or cytochrome c are normalized to β-actin (cytosolic fraction) or COX IV (mitochondrial fraction), respectively. Values are mean±SD of 3 independent experiments. *P<0.01; comparing the percentage of Bax or cytochrome c in cytosol or mitochondria in B-CLL cells with or without CpG-B ODNs or IL-10. (B) Representative results of intracellular staining of Bax activation and cytochrome c release (shaded histogram) at day 3 cultures in B-CLL cells with or without CpG-B ODNs or IL-10, indicated with MFI number, and overlaid with isotype control (unshaded histogram).

FIGS. 4A-4C IL10RA gene deletion and IL-10R1 S138G homozygous mutation are detected in CpG-resistant but not CpG-sensitive B-CLL cells. (A) Scheme of MLL and IL10RA genes position in chromosome 11q23.3 (left panel), and FISH results of MLL and IL10RA genes in B-CLL cells with or without MLL and IL10RA genes deletion (right penal). (B) Scheme of the IL-10R1 S138G mutation site described in the IL-10R1 protein sequence and IL10RA mRNA sequence (left panel). Three IL-10R1 S138G genotypes of B-CLL cells by Bi-PASA (right panel). Lane 1, Low DNA Mass™ Ladder (Invitrogen); lane 2 and 3, homozygous wild-type (AA); lane 4 and 5 homozygous mutant (GG); lane 6 and 7, heterozygote (AG). Numbers on the right indicate the size of the amplicons. Data are representative results from six B-CLL samples. (C) IL-10R1 expression (left panel) and IL-10 binding (right panel) results from 15 IL-10R1 S138G wild-type (WT), 7 IL-10R1 S138G homozygous mutant and 2 IL10RA gene deletion individuals of B-CLL samples. Each dot represents an individual patient sample and the horizontal bar represents the median level. *P<0.05, comparing MFI with wild-type group of B-CLL cells.

FIGS. 5A-5B. CpG-B ODN fails to induce STAT1/3 activation in B-CLL cells with IL10RA gene deletion or mutation. (A) Western blot of CpG-B ODNs-induced tyrosine- or serine-phosphorylated forms of STAT1 expression in B-CLL cells from 5 CpG-sensitive wild-type, 3 IL-10R1 S138G homozygous variant and 2 IL10RA gene deletion B-CLL samples at 24-hour cultures (left panel). Data are densitometrically assessed and presented as the mean±SD in the adjacent bars diagram (right panel). (B) Western blot of CpG-B ODNs-induced tyrosine- or serine-phosphorylated forms of STAT3 expression in B-CLL cells from 5 CpG-sensitive wild-type, 3 IL-10R1 S138G variant and 2 IL10RA gene deletion CLL samples at 24-hour cultures. Data are densitometrically assessed and presented as the mean±SD in the adjacent bars diagram (right panel). *P<0.01, comparing pTyr701-STAT1 or pTyr705-STAT3 expression in B-CLL cells with or without CpG-B ODNs stimulation. **P<.0.01, comparing pTyr701-STAT1 or pTyr705-STAT3 expression in B-CLL cells from IL10RA gene deletion or mutation with wild-type CLL samples following CpG-B ODN stimulation.

FIGS. 6A-6B. IL-10 induced STAT1/3 activation are abolished or reduced in B-CLL cells with IL10RA gene deletion or mutation. (A) Western blot of IL-10 induced tyrosine- or serine-phosphorylated forms of STAT1 expression in B-CLL cells from 5 CpG-sensitive wild-type, 3 IL-10R1 S138G variant and 2 IL10RA gene deletion B-CLL samples at 24-hour cultures (left panel). Data are densitometrically assessed and presented as the mean±SD in the adjacent bar diagram (right panel). (B) Western blot of IL-10-induced tyrosine- or serine-phosphorylated forms of STAT3 expression in B-CLL cells from 5 CpG-sensitive wild-type, 3 IL-10R1 S138G variant and 2 IL10RA gene deletion B-CLL samples at 24-hour cultures (left panel). Data are densitometrically assessed and presented as the mean±SD in the adjacent bar diagram (right panel). *P<0.01, comparing pTyr701-STAT1 or pTyr705-STAT3 expression in B-CLL cells with or without IL-10 stimulation. **P<0.01, comparing pTyr705-STAT3 expression in B-CLL cells from IL10RA gene deletion or mutation with wild-type CLL samples following IL-10 stimulation.

FIGS. 7A and 7B. FIG. 7A provides the amino acid sequence for interleukin-10 receptor subunit alpha precursor [Homo sapiens]. NCBI link: http://www.ncbi.nlm.nih.gov/protein/222136575. FIG. 7B provides the nucleic acid sequence for Homo sapiens interleukin 10 receptor, alpha (IL10RA), transcript variant 1, mRNA. NCBI link: http://www.ncbi.nlm.nih.gov/nuccore/NM_001558.3

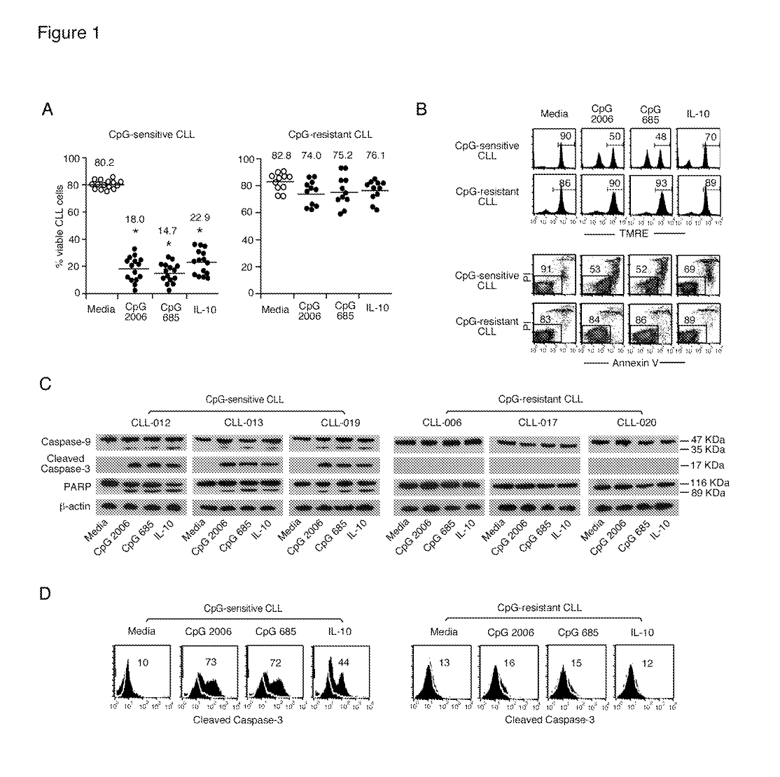

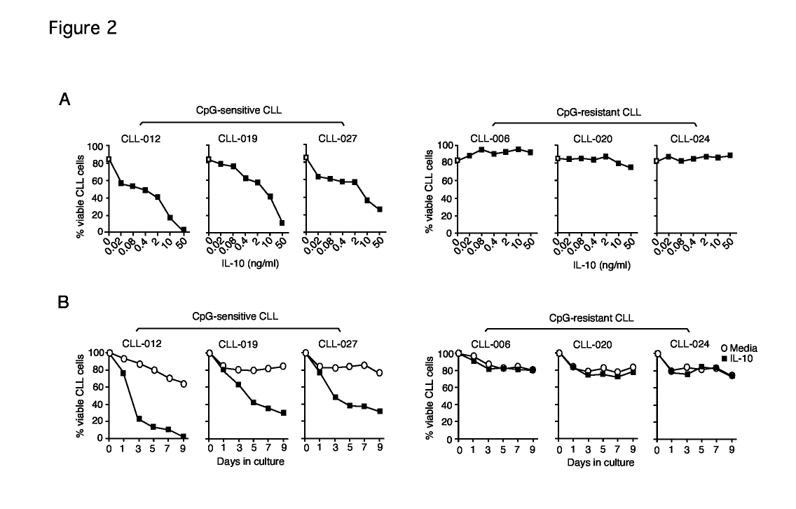

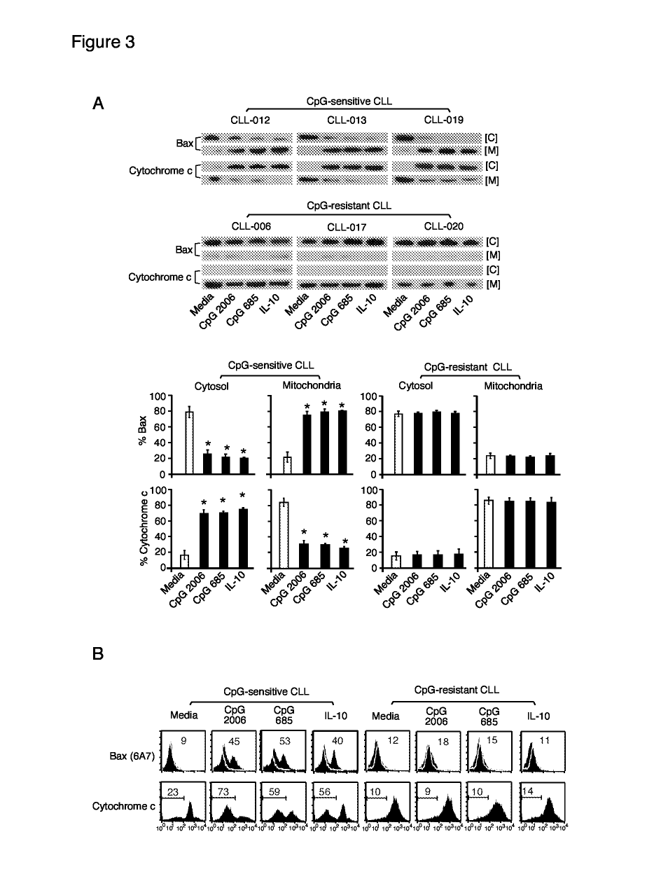

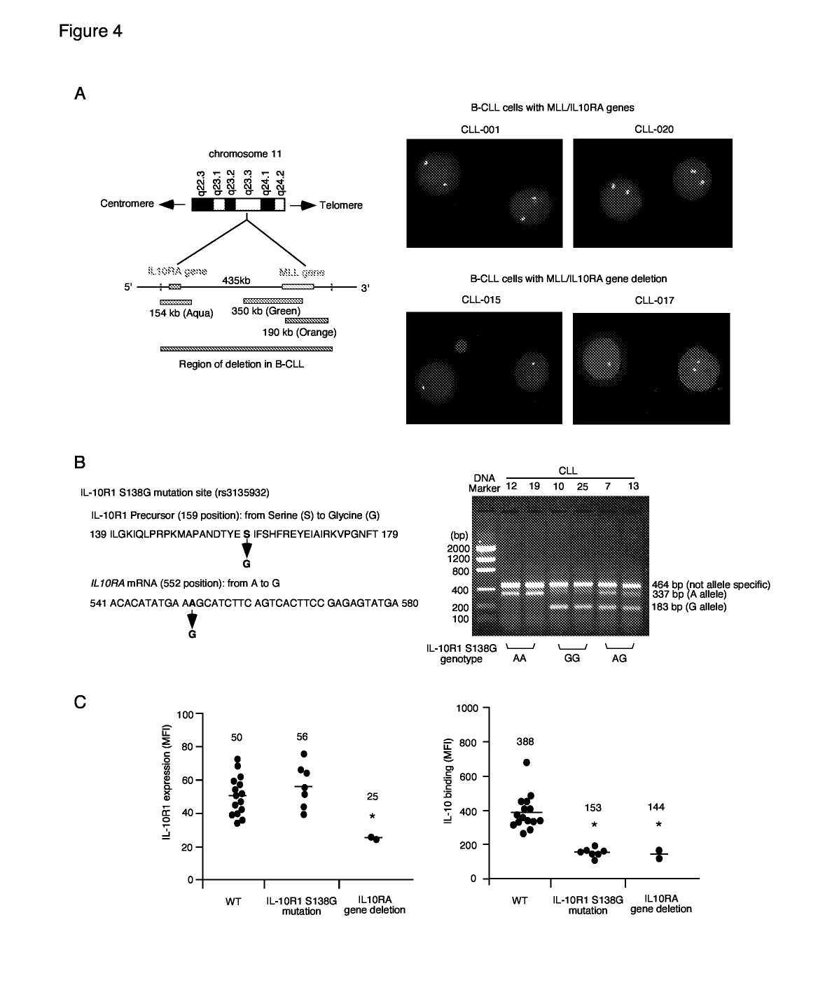

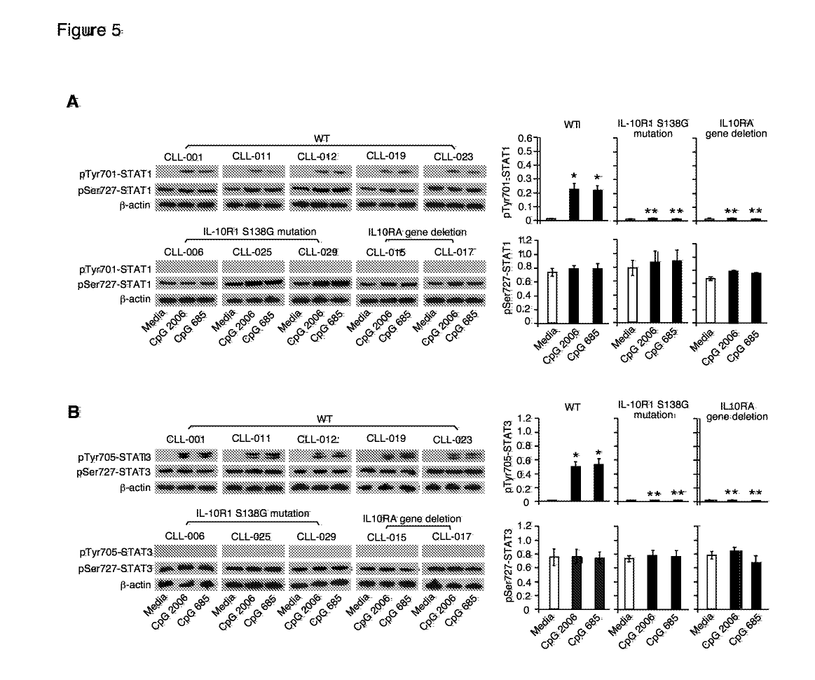

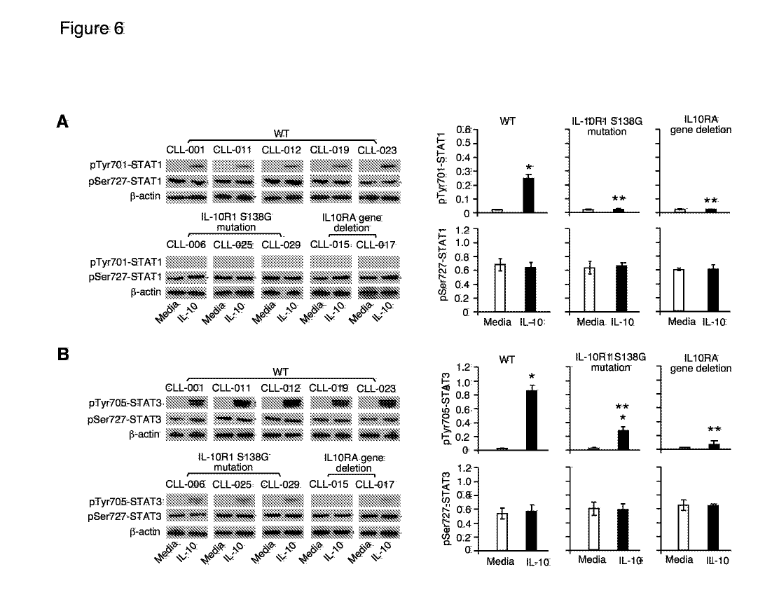

### DETAILED DESCRIPTION OF THE INVENTION

The inventor discovered that deletion and mutation of the IL10RA gene on chromosome 11 q23 plays an important role in the development of drug-resistance in CLL patients. It has been found that it can serve as a pharmacogenomic biomaker classifier and can be used to select, stratify, or exclude patients with B cell malignancies for certain therapies. It can also be used to predict clinical outcomes. This important new finding can lead to the development of companion diagnostics not only in the treatment of human B cell malignancies but also possibly in immune-based therapies of other cancer, autoimmune diseases, and organ transplantations.

Targeting Toll-like receptor 9 (TLR9) expressed on human chronic lymphocytic leukemia (B-CLL) cells with TLR9 agonist drugs CpG-B oligodeoxynucleotides (CpG ODN) led to IL-10-induced tyrosine phosphorylation of signal transducers and activators of transcription (STATs) and apoptosis of B-CLL cells from majority CLL patients. Here, the molecular mechanisms underlying a subset of CLL patients whose B-CLL cells are resistant to CpG-induced apoptosis was shown. Primary B-CLL cells from 15/15 CpG-sensitive samples were induced into apoptosis by either CpG-B ODNs or IL-10 in a treatment time and dose-dependent manner, with significantly increased pTyr701-STAT1/pTyr705-STAT3 expression and apoptosis via the mitochondrial pathway. No IL10RA gene deletion or IL-10R1 S138G homozygous mutation was detected. 13/15 patients were IL-10R1 S138G AA wildtypes and 2/15 patients were AG heterozygotes. In contrast, CpG-B ODNs or IL-10 failed to induce apoptosis in 11/11 CpG-resistant B-CLL cells. 2/11 CLL samples had IL10RA gene deletion and 7/11 had IL-10R1 S138G homozygous mutation. IL10RA gene deletion significantly decreased the IL-10R1 expression. Both IL10RA gene deletion and mutation decreased the IL-10 binding, abolished or reduced pTyr701-STAT1/pTyr705-STAT3 expression in B-CLL cells treated with CpG-B ODNs or IL-10. These findings demonstrate that deletion and mutation of the IL10RA gene on chromosome 11q23 averted CpG-B ODN-induced apoptosis in B-CLL cells, which serves as pharmacogenomics biomarker classifiers to predict, select, or stratify CLL patients to CpG-B ODN treatment.

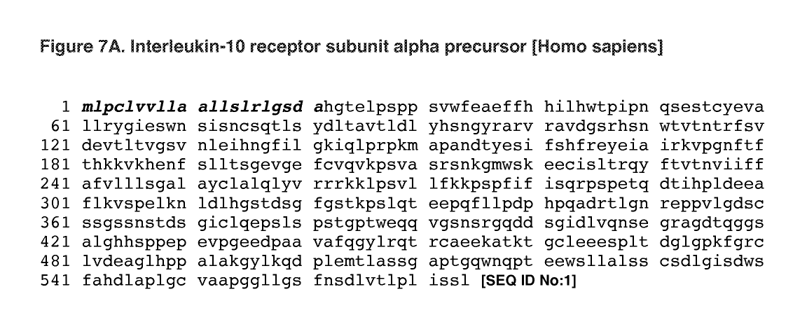

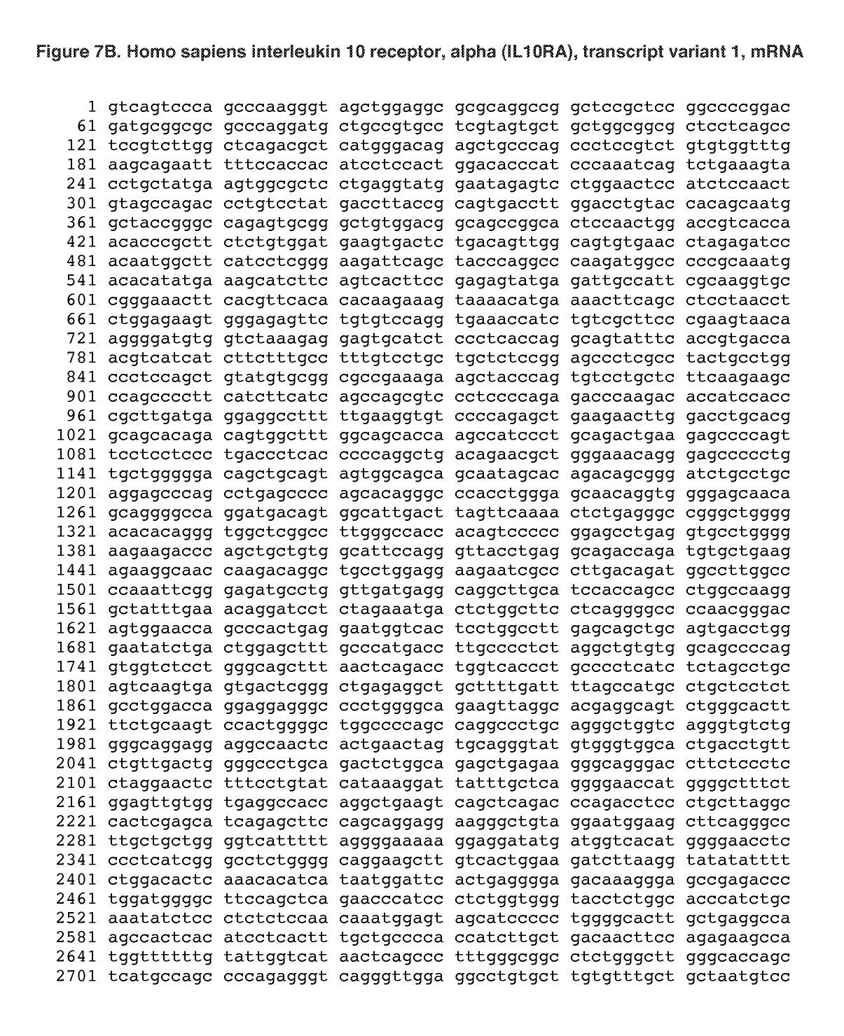

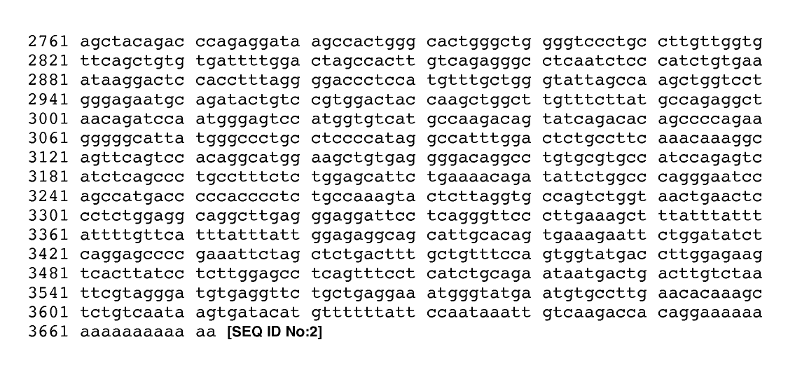

The IL-10R1 S138G mutation happens at 138 amino acid position of IL-10R1 protein where Serine (S) was replaced by Glycine (G) (NCBI Reference SNP Cluster Report: rs3135932), or 159 amino acid position of the IL-10R1 precursor that includes a 21-amino acid signal peptide at the beginning (FIG. 7A). The mutation in IL10RA mRNA happens at 536 (IL10RA) or 552 (IL10RA precursor) position (FIG. 7B, NCBI Reference SNP Cluster Report: rs3135932), where Adenine (A) was replaced by Guanine (G). The amino acid (IL-10R1) and nucleic acid sequence (IL10RA) and their links at NCBI are listed in FIG. 7 legend.

Patients that have drug-resistant CLL are generally homozygous for the mutant IL10RA. An “allele” is a variant form of a particular gene. For example, the present invention relates, inter alia, to the discovery that some alleles of the IL10RA gene cause drug-resistant CLL in mammals. A “IL10RA allele” refers to a normal allele of the IL10RA locus as well as an allele carrying a variation(s) that predispose a mammal to develop drug-resistant CLL. The coexistence of multiple alleles at a locus is known as “genetic polymorphism.” Any site at which multiple alleles exist as stable components of the population is by definition “polymorphic.” An allele is defined as polymorphic if it is present at a frequency of at least 1% in the population. A “single nucleotide polymorphism (SNP)” is a DNA sequence variation that involves a change in a single nucleotide.

The terms “protein,” “peptide” and “polypeptide” are used interchangeably herein.

The invention encompasses isolated or substantially purified nucleic acid compositions. In the context of the present invention, an “isolated” or “purified” DNA molecule is a DNA molecule that, by human intervention, exists apart from its native environment and is therefore not a product of nature. An isolated DNA molecule may exist in a purified form or may exist in a non-native environment. For example, an “isolated” or “purified” nucleic acid molecule, or portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences and proteins or partial-length proteins encoded thereby are also encompassed by the present invention.

By “fragment” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment” means a sequence having, for example, at least 80 nucleotides, at least 150 nucleotides, or at least 400 nucleotides. If not employed for expressing, a “portion” or “fragment” means, for example, at least 9, 12, 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Synthetic” polynucleotides are those prepared by chemical synthesis.

“Recombinant DNA molecule” is a combination of DNA sequences that are joined together using recombinant DNA technology and procedures used to join together DNA sequences as described, for example, in Sambrook and Russell (2001).

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, such as IL-10R1, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

A “mutant” IL-10R1 refers to the protein or fragment thereof that is encoded by a IL10RA gene having a mutation, e.g., such as might occur at the IL10RA locus. A mutation in IL10RA may leads to drug-resistant CLL in a mammal homozygous for the allele. Mutations in IL10RA may cause drug-resistant CLL in a mammal homozygous for the mutant IL10RA allele, e.g., a mammal homozygous for a mutation leading to a mutant gene product such as a substitution or deletion mutation in IL10RA, such as that designated herein as S159G.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (see the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When using BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the World Wide Web at ncbi.nlm.nih.gov. Alignment may also be performed manually by visual inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by a BLAST program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, or at least 80%, 90%, or even at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%; at least 80%, 81%, 82%, 83%, 84%, 85%, 86%, 87%, 88%, or 89%; or at least 90%, 91%, 92%, 93%, or 94%; or even at least 95%, 96%, 97%, 98% or 99% sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl:

Tₘ81.5° C.+16.6(log M)+0.41(%GC)−0.61(% form)−500/L

where M is the molarity of monovalent cations, %GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1× SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. Guidance as to appropriate amino acid substitutions that do not affect biological activity of the protein of interest are well known in the art. Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally-occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions, deletions, or additions that alter, add or delete a single amino acid or a small percentage of amino acids (typically less than 5%, more typically less than 1%) in an encoded sequence are “conservatively modified variations.”

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Genome” refers to the complete genetic material of an organism.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

Nucleic Acid Probes of the Invention

Sources of nucleotide sequences from which the present nucleic acid molecules useful as probes can be obtained include any prokaryotic or eukaryotic source. For example, they can be obtained from a mammalian cellular source.

As discussed above, the terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule that is complementary or hybridizes to a sequence in a gene of interest, i.e., a nucleic acid sequence encoding IL-10R1, and remains stably bound under stringent conditions (as defined by methods well known in the art). Thus, the RNA or DNA is “isolated” in that it is free from at least one contaminating nucleic acid with which it is normally associated in the natural source of the RNA or DNA and in one embodiment of the invention is substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

Nucleic Acid Amplification Methods

According to the methods of the present invention, the amplification of DNA present in a physiological sample may be carried out by any means known to the art. Examples of suitable amplification techniques include, but are not limited to, polymerase chain reaction (including, for RNA amplification, reverse-transcriptase polymerase chain reaction), ligase chain reaction, strand displacement amplification, transcription-based amplification, self-sustained sequence replication (or “3SR”), the Qβ replicase system, nucleic acid sequence-based amplification (or “NASBA”), the repair chain reaction (or “RCR”), and boomerang DNA amplification (or “BDA”).

The bases incorporated into the amplification product may be natural or modified bases (modified before or after amplification), and the bases may be selected to optimize subsequent electrochemical detection steps.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized that is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques. Where the nucleic acid to be amplified is RNA, amplification may be carried out by initial conversion to DNA by reverse transcriptase in accordance with known techniques.

Strand displacement amplification (SDA) may be carried out in accordance with known techniques. For example, SDA may be carried out with a single amplification primer or a pair of amplification primers, with exponential amplification being achieved with the latter. In general, SDA amplification primers comprise, in the 5′ to 3′ direction, a flanking sequence (the DNA sequence of which is noncritical), a restriction site for the restriction enzyme employed in the reaction, and an oligonucleotide sequence (e.g., an oligonucleotide probe of the present invention) that hybridizes to the target sequence to be amplified and/or detected. The flanking sequence, which serves to facilitate binding of the restriction enzyme to the recognition site and provides a DNA polymerase priming site after the restriction site has been nicked, is about 15 to 20 nucleotides in length in one embodiment. The restriction site is functional in the SDA reaction. The oligonucleotide probe portion is about 13 to 15 nucleotides in length in one embodiment of the invention.

Ligase chain reaction (LCR) is also carried out in accordance with known techniques. In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

In one embodiment of the invention, the IL10RA gene is amplified by PCR using primers based on the known sequence. The amplified exons are then sequenced using automated sequencers. In this manner, the IL10RA gene from a mammal suspected of having drug-resistant CLL is sequenced. Examples of such mutations include those in the IL10RA DNA. For example, one mutation is the A to G substitution at nucleotide base 552 of SEQ ID NO:2.

According to the diagnostic method of the present invention, alteration within the wild-type IL10RA locus is detected. “Alteration of a wild-type gene” encompasses all forms of mutations including deletions, insertions and point mutations in the coding and noncoding regions. Deletions may be of the entire gene or of only a portion of the gene. Point mutations may result in stop codons, frameshift mutations or amino acid substitutions. Point mutational events may occur in regulatory regions, such as in the promoter of the gene, leading to loss or diminution of expression of the mRNA. Point mutations may also abolish proper RNA processing, leading to loss of expression of the IL10RA gene product, or to a decrease in mRNA stability or translation efficiency. Mammals predisposed to or have drug-resistant CLL are generally homozygous for the mutated alleles.

Diagnostic techniques that are useful in the methods of the invention include, but are not limited to direct DNA sequencing, PFGE analysis, allele-specific oligonucleotide (ASO), dot blot analysis and denaturing gradient gel electrophoresis, and are well known to the artisan.

There are several methods that can be used to detect DNA sequence variation. Direct DNA sequencing, either manual sequencing or automated fluorescent sequencing can detect sequence variation. Another approach is the single-stranded conformation polymorphism assay (SSCA). This method does not detect all sequence changes, especially if the DNA fragment size is greater than 200 bp, but can be optimized to detect most DNA sequence variation. The reduced detection sensitivity is a disadvantage, but the increased throughput possible with SSCA makes it an attractive, viable alternative to direct sequencing for mutation detection on a research basis. The fragments that have shifted mobility on SSCA gels are then sequenced to determine the exact nature of the DNA sequence variation. Other approaches based on the detection of mismatches between the two complementary DNA strands include clamped denaturing gel electrophoresis (CDGE), heteroduplex analysis (HA) and chemical mismatch cleavage (CMC). Once a mutation is known, an allele specific detection approach such as allele specific oligonucleotide (ASO) hybridization can be utilized to rapidly screen large numbers of other samples for that same mutation. Such a technique can utilize probes which are labeled with gold nanoparticles to yield a visual color result.

Detection of point mutations may be accomplished by molecular cloning of the IL10RA allele(s) and sequencing the allele(s) using techniques well known in the art. Alternatively, the gene sequences can be amplified directly from a genomic DNA preparation from mammalian tissue, using known techniques. The DNA sequence of the amplified sequences can then be determined.

There are six well known methods for a more complete, yet still indirect, test for confirming the presence of a mutant allele: 1) single stranded conformation analysis (SSCA); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6) allele-specific PCR. For allele-specific PCR, primers are used which hybridize at their 3′ ends to a particular IL10RA mutation. If the particular mutation is not present, an amplification product is not observed. Amplification Refractory Mutation System (ARMS) can also be used. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Other techniques for detecting insertions and deletions as known in the art can be used.

In the first three methods (SSCA, DGGE and RNase protection assay), a new electrophoretic band appears. SSCA detects a band that migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between mutant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of samples. An example of a mismatch cleavage technique is the RNase protection method. In the practice of the present invention, the method involves the use of a labeled riboprobe that is complementary to the wild-type IL10RA gene coding sequence. The riboprobe and either mRNA or DNA isolated from the tumor tissue are annealed (hybridized) together and subsequently digested with the enzyme RNase A that is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the IL10RA mRNA or gene but can be a segment of either. If the riboprobe comprises only a segment of the IL10RA mRNA or gene, it will be desirable to use a number of these probes to screen the whole mRNA sequence for mismatches.

In similar fashion, DNA probes can be used to detect mismatches, through enzymatic or chemical cleavage. Alternatively, mismatches can be detected by shifts in the electrophoretic mobility of mismatched duplexes relative to matched duplexes. With either riboprobes or DNA probes, the cellular mRNA or DNA that might contain a mutation can be amplified using PCR before hybridization.

Nucleic acid analysis via microchip technology is also applicable to the present invention.

DNA sequences of the IL10RA gene that have been amplified by use of PCR may also be screened using allele-specific probes. These probes are nucleic acid oligomers, each of which contains a region of the IL10RA gene sequence harboring a known mutation. For example, one oligomer may be about 30 nucleotides in length, corresponding to a portion of the IL10RA gene sequence. By use of a battery of such allele-specific probes, PCR amplification products can be screened to identify the presence of a previously identified mutation in the IL10RA gene. Hybridization of allele-specific probes with amplified IL10RA sequences can be performed, for example, on a nylon filter. Hybridization to a particular probe under stringent hybridization conditions indicates the presence of the same mutation in the tissue as in the allele-specific probe.

Alteration of wild-type IL10RA genes can also be detected by screening for alteration of wild-type IL-10R1 protein, or a portion of the IL-10R1 protein. For example, monoclonal antibodies immunoreactive with IL-10R1 (or to a specific portion of the IL-10R1 protein) can be used to screen a tissue. Lack of cognate antigen would indicate a mutation. Antibodies specific for products of mutant alleles could also be used to detect mutant IL10RA gene product. Such immunological assays can be done in any convenient formats known in the art. These include Western blots, immunohistochemical assays and ELISA assays. Any means for detecting an altered IL-10R1 protein can be used to detect alteration of wild-type IL10RA genes. Functional assays, such as protein binding determinations, can be used. In addition, assays can be used that detect IL-10R1 biochemical function. Finding a mutant IL10RA gene product indicates alteration of a wild-type IL10RA gene.

Mutant IL10RA genes or gene products can be detected in a variety of physiological samples collected from a mammal. Examples of appropriate samples include a cell sample, such as a blood cell, e.g., a lymphocyte, a peripheral blood cell; a sample collected from the spinal cord; a tissue sample such as cardiac tissue or muscle tissue, e.g., cardiac or skeletal muscle; an organ sample, e.g., liver or skin; a hair sample, especially a hair sample with roots; a fluid sample, such as blood.

The methods of diagnosis of the present invention are applicable to any disease in which IL10RA has a role. The diagnostic method of the present invention is useful, for example, for veterinarians, Breed Associations, or individual breeders, so they can decide upon an appropriate course of treatment, and/or to determine if an animal is a suitable candidate as a broodmare or sire.

Oligonucleotide Probes

As noted above, the method of the present invention is useful for detecting the presence of a polymorphism in mammalian DNA, in particular, the presence of a A to G nucleotide substitution at position 552 in the coding sequence of IL10RA (SEQ ID NO:2). This substitution results in the replacement of a Serine (S) amino acid by a Glysine (G) in the protein (SEQ ID NO:1).

Primer pairs are useful for determination of the nucleotide sequence of a particular IL10RA allele using PCR. The pairs of single-stranded DNA primers can be annealed to sequences within or surrounding the IL10RA gene in order to prime amplify DNA synthesis of the IL10RA gene itself. Allele-specific primers can also be used. Such primers anneal only to particular IL10RA mutant alleles, and thus will only amplify a product in the presence of the mutant allele as a template.

The first step of the process involves contacting a physiological sample obtained from a mammal, which sample contains nucleic acid, with an oligonucleotide probe to form a hybridized DNA. The oligonucleotide probes that are useful in the methods of the present invention can be any probe comprised of between about 4 or 6 bases up to about 80 or 100 bases or more. In one embodiment of the present invention, the probes are between about 10 and about 20 bases.

The primers themselves can be synthesized using techniques that are well known in the art. Generally, the primers can be made using oligonucleotide synthesizing machines that are commercially available. Given the sequence of the IL10RA coding sequence as set forth in SEQ ID NO:1, design of particular primers is well within the skill of the art.

Oligonucleotide probes may be prepared having any of a wide variety of base sequences according to techniques that are well known in the art. Suitable bases for preparing the oligonucleotide probe may be selected from naturally occurring nucleotide bases such as adenine, cytosine, guanine, uracil, and thymine; and non-naturally occurring or “synthetic” nucleotide bases such as 7-deaza-guanine 8-oxo-guanine, 6-mercaptoguanine, 4-acetylcytidine, 5-(carboxyhydroxyethyl)uridine, 2′-O-methylcytidine, 5-carboxymethylamino-methyl-2-thioridine, 5-carboxymethylaminomethyluridine, dihydrouridine, 2′-O-methylpseudouridine, β,D-galactosylqueosine, 2′-O-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylamninomethyluridine, 5-methoxyaminomethyl-2-thiouridine, β,D-mannosylqueosine, 5-methloxycarbonylmethyluridine, 5-methoxyuridine, 2-methyltio-N6-isopentenyladenosine, N-((9-β-D-ribofuranosyl-2-methylthiopurine-6-yl)carbamoyl)threonine, N-((9-β-D-ribofuranosylpurine-6-yl)N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-Methylurdine, N-((9-beta-D-ribofuranosylpurine-6-yl)carbamoyl)threonine, 2′-O-methyl-5-methyluridine, 2′-O-methylurdine, wybutosine, and 3-(3-amino-3-carboxypropyl)uridine. Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the internucleotide bridging phosphate residues are modified phosphates, such as methyl phosphonates, methyl phosphonotlioates, phosphoroinorpholidates, phosphoropiperazidates and phosplioramidates (for example, every other one of the internucleotide bridging phosphate residues may be modified as described). The oligonucleotide may be a “peptide nucleic acid” such as described in Nielsen et al., Science, 254, 1497-1500 (1991).

The only requirement is that the oligonucleotide probe should possess a sequence at least a portion of which is capable of binding to a known portion of the sequence of the DNA sample.

It may be desirable in some applications to contact the DNA sample with a number of oligonucleotide probes having different base sequences (e.g., where there are two or more target nucleic acids in the sample, or where a single target nucleic acid is hybridized to two or more probes in a “sandwich” assay).

The nucleic acid probes provided by the present invention are useful for a number of purposes. The probes can be used to detect PCR amplification products.

Hybridization Methodology

The DNA (or nucleic acid) sample may be contacted with the oligonucleotide probe in any suitable manner known to those skilled in the art. For example, the DNA sample may be solubilized in solution, and contacted with the oligonucleotide probe by solubilizing the oligonucleotide probe in solution with the DNA sample under conditions that permit hybridization. Suitable conditions are well known to those skilled in the art. Alternatively, the DNA sample may be solubilized in solution with the oligonucleotide probe immobilized on a solid support, whereby the DNA sample may be contacted with the oligonucleotide probe by immersing the solid support having the oligonucleotide probe immobilized thereon in the solution containing the DNA sample.

### EXAMPLE 1

### Deletion and Mutation of IL10RA Gene on Chromosome 11q23 avert CpG-B Oligodeoxynucleotides-Induced Apoptosis of Human Chronic Lymphocytic Leukemia B Cells

Targeting TLR9 expressed on human CLL cells with CpG-B oligodeoxynucleotides leads to IL-10-induced tyrosine phosphorylation of STATs and apoptosis of B-CLL cells. However, B-CLL cells from a small subset of patients were resistant to CpG-B ODN treatment. Here, we investigated the molecular mechanism by which B-CLL cells are sensitive or resistant to CpG-B ODN treatment.

Purified CD19⁺CD23⁺CD5⁺ primary B-CLL cells were cultured for 5 days with/without CpG-B ODN (CpG 2006, CpG 685) or rh-IL-10. B-CLL cell apoptosis were determined by viable cell counts, Annexin V/PI and TMRE staining, western blot and intracellular staining of the activation/cleavage of caspases, PARP, Bax translocation and cytochrome c release, IL-10R1 expression and IL-10 binding by flow cytometry, IL10RA gene deletion by FISH, IL-10R1 S138G mutation by Bi-PASA, The tyrosine or serine phosphorylation of STATs by Western blot.

Fifteen CpG-sensitive and 11 CpG-resistant primary CLL samples were comparatively studied. B-CLL cells from 15/15 CpG-sensitive samples were induced into apoptosis by either CpG-B ODNs or IL-10 in a treatment time and dose-dependent manner. Both CpG-B ODNs and IL-10 significantly increased pTyr701-STAT1/pTyr705-STAT3 expression and induced apoptosis via the mitochondrial apoptotic pathway in 15 primary B-CLL cells. No IL-10RA gene deletions were detected, 13/15 patients were IL-10R1 S138G AA wildtypes and 2/15 patients were AG heterozygotes. In contrast, CpG-B ODNs or IL-10 failed to induce apoptosis in 11/11 CpG-resistant B-CLL cells. Interesting, 2/11 CLL samples had IL10RA genes deletion and 7/11 had IL-10R1 S138G GG homozygote mutation. IL10RA gene deletion significantly decreased the IL-10R1 expression; both IL10RA gene deletion and mutation significantly decreased the IL-10 binding, abolished or reduced CpG-B ODNs or IL10-induced pTyr701-STAT1/pTyr705-STAT3 expression in B-CLL cells.

Deletion and mutation of IL10RA gene on chromosome 11q23 averts CpG-B ODN-induced apoptosis of human B-CLL cells, which may serve as biomarker to predict sensitivity or resistance of CLL to CpG-B ODN treatment.

### METHODS

Cell Preparations

Blood samples from 26 B-CLL patients were drawn after obtaining written informed consent approved by the University of Minnesota Institutional Review Board. Peripheral blood mononuclear cells (PBMCs) were isolated by Ficoll-Paque density gradient centrifugation. B-CLL cells in PBMCs were purified using B Cell Isolation Kit (B-CLL, Miltenyi Biotec) to >95% of CD5⁺CD19⁺CD23⁺cells.

CpG Oligodeoxynucleotides

Phosphorothioated unmethylated CpG-B ODNs (2006: 5′-tcgtcgttttgtcgttttgtcgtt-3′, 685: 5′-tcgtcgacgtcgttcgttctc-3′) were purchased from Intergrated DNA Technologies. CpG-B ODNs were resuspended in TE buffer, diluted in PBS, and tested at a final concentration of 5 μg/ml or as indicated.

B-CLL Cell Cultures

To determine CpG-B ODNs or IL-10 treatment time or IL-10 doses on apoptosis induction, B-CLL cells (2×10⁵/well) were cultured in media with or without CpG-B ODNs or IL-10 (R&D Systems; tested at a final concentration of 10 ng/ml) for up to 9 days or at different doses of IL-10 (0.02 to 50 ng/ml) for 5 days in 96-well plates. Several wells of B-CLL cells from each culture condition were harvested at the indicated time points, counted, and stained with tetramethyl-rhodamine ethylester (TMRE, Invitrogen) or Annexin V-FITC Apoptosis Detection Kit I (BD Biosciences). Viable B-CLL cell number was calculated by multiplying total cell counts with the percentage of TMRE-positive cells of each culture condition. B-CLL cells cultured with or without CpG-B ODNs or IL-10 were used to determine STATs expression at 24-hour cultures, Bax translocation and cytochrome c release at day 3 cultures, or cleavage of caspases, poly (ADP-ribose) polymerase (PARP) at day 5 cultures using western blot or intracellular staining.

### IL-10 Binding

IL-10 binding with B-CLL cells was determined using Flurokine® Biotinylated Human Interleukin 10 Kit (R&D Systems) per the protocol. 25 μl (1×10⁵) purified B-CLL cells were incubated with 10 μl biotinylated negative control reagents or biotinylated IL-10 for 1-hour at 2-8° C., then incubated with 10 μl of avidin-FITC for a further 30 minutes at 2-8° C. in the dark. Wash the cells twice with 2 ml of 1×RDF buffer 2 times. Add 200 μl 1×RDF buffer into each tube and do flow cytometry analysis.

Flow Cytometry

Cell surface markers were analyzed by staining with fluorescent Abs against CD5, CD19, CD23, CD45, IL-10R1 or isotype control Ab (BD Bioscience). Apoptotic B-CLL cells were assessed by TMRE or Annexin V/propidium iodide (PI) staining Mean fluorescence intensity (MFI) and positive cell percentages were determined.

For intracellular staining, 1×10⁶ B-CLL cells were fixed with 2% formaldehyde for 30 minutes at 4° C. Cells were incubated with anti-cleaved caspase-3 (Cell Signaling Technology) or anti-Bax (clone 6A7, Santa Cruz Biotechnology) at 1:20 dilution in 2% FBS, 1% Saponin in PBS at 4° C. for 30 minutes, followed by incubated with goat-anti rabbit IgG PE (for cleaved caspase-3, Santa Cruz Biotechnology) or goat-anti mouse IgG PE (for Bax 6A7, Santa Cruz Biotechnology) at 1:100 at 4° C. respectively for 30 minutes.

For the immunodetection of cytochrome c release, 1×10⁶ B-CLL cells were permeabilized with digitonin (50 μg/ml in cold PBS, 100 mM KCl ) for 30 seconds to 1 minute on ice, wash 3 times with PBS, then fixed with 4% formaldehyde for 20 minutes at room temperature. Cells were incubated with anti-cytochrome c antibody (Santa Cruz Biotechnology) at 1:20 dilution for 1 hour with blocking buffer (3% BSA, and 0.05% saponin), followed by incubated with goat anti-rabbit PE at 1:100 dilution for 30 minutes at room temperature.

Fluorescence in-Situ Hybridization (FISH)

Purified B-CLL cells were harvested according to standard cytogenetic protocol for direct FISH. Cytogenetic abnormalities in each patient were evaluated by FISH using Vysis LSI ATM, LSI D135319, LSI p53, LSI MLL dual color probes (Abbott Molecular) and IL10RA probes (RP11-36O11, Empire Genomics). The 3-color FISH was performed with DNA probes for MLL gene (Dual Color Break apart-Spectrum Green and Orange) mapped to the sequences 5′ and 3′ of the common breakpoint region, and IL10RA gene (Spectrum Aqua) per the manufacturer protocol. FISH signals were visualized using a fluorescent imaging workstation (Applied Imaging) and images were captured using the CytoVision software (Applied Imaging). At least 200 interphase cells were examined and the number of abnormal cells was expressed as a percentage of the total number scored.

Detection of IL-10R1 S138G Mutation

Genomic DNA was extracted from purified B-CLL cells using PureLink™ Genomic DNA Mini Kit (Invitrogen). IL-10R1 S138G genotypes were detected by Bidirectional PCR amplification of specific alleles (Bi-PASA) with IL-10R1 S138G Mutation Detection Kit (ADC laboratories) per manufacture protocol. The PCR products are 464 by (not allele specific product), 337 by (wild-type allele, A allele) and 183 by (mutant allele, G allele). The IL-10R1 S138G genotypes are shown as AA homozygous wild-type, GG homozygous mutant, and AG heterozygote, respectively.

Subcellular Fractionation

Purified B-CLL cells cultured with or without CpG-B ODNs or IL-10 for 3 days were lysed in isotonic buffer (200 mM mannitol, 70 mM sucrose, 1 mM EDTA, 10 mM HEPES, pH 6.9 and 1mM DTT) containing protease inhibitors (10 μg/ml Aprotinin, 10 μg/ml Leupeptin). The cells were passed through a 26-gauge needle fitted to a syringe, and centrifuged at 1000×g for 10 minutes at 4° C. to remove unbroken cells and nuclei. The resulting supernatants were further centrifuged at 10,000×g for 30 minutes at 4° C. to obtain the heavy membrane pellet enriched for mitochondria, and the supernatant as crude cytosol. The heavy membrane pellet was washed once with isotonic buffer, resuspended in RIPA lysis buffer (10 mM Tris-HCl pH 8.0, 150 mM NaCl, 1 mM EDTA, 0.5% Sodium deoxycholate, 0.1% SDS, 1% Nonidet P-40) containing protease inhibitors. The debris was removed by a brief centrifugation.

Western Blots

Protein lysates were prepared from purified B-CLL cells cultured with or without CpG-B ODNs or IL-10 for the indicated time. Western blot was performed using primary antibodies specific for tyrosine- or serine-phosphorylated STAT1 or STAT3 (Santa Cruz Biotechnology), cleaved caspase-3, caspase-9 or PARP (Cell Signaling Technology), respectively. Subcellular fractions were probed with anti-Bax (N20), anti-cytochrome c antibodies (Santa Cruz Biotechnology). Then probed with horseradish peroxidase conjugated secondary antibody (Santa Cruz Biotechnology), and visualized by Pierce ECL Western Blotting Substrate (Thermo Fisher Scientific Inc). Blots were reprobed with anti-β-actin antibody (Santa Cruz Biotechnology) as a loading control for whole cell lysate or cytosolic fraction, or anti-cytochrome c oxidase subunit IV (COX IV, Santa Cruz Biotechnology) as a loading control for the mitochondrial fraction. Signals intensity was quantified using Fluorochem 8000 imaging system (Alpha Innotech).

Data Analysis

Statistical analysis of the results was performed by one-way analysis of variance (ANOVA) test, with Scheffe post hoc testing to determine where differences existed. Values of P<0.05 were considered significant.

### SUMMARY

B-CLL cells from a subset of CLL patients are found to be resistant to CpG-B ODNs or IL-10 induced apoptosis. IL-10 induces apoptosis is drug-dose and treatment time dependent in CpG-sensitive but not CpG-resistant B-CLL cells. CpG-B ODNs and IL-10 induce apoptosis via mitochondrial apoptotic pathway in CpG-sensitive but not CpG-resistant B-CLL cells. Deletion of IL10RA gene and IL-10R1 S138G homozygous mutation were found in CpG-resistant but not CpG-sensitive CLL samples. IL10RA gene deletion and IL-10R1 S138G mutation affect IL-10R1 expression on and IL-10 binding to B-CLL cells. CpG-B ODNs fail to induce STAT1/3 activation in B-CLL cells with IL10RA gene deletion or IL-10R1 S138G homozygous mutation. IL-10-induced STAT1/3 activation are abolished or reduced in B-CLL cells with IL10RA gene deletion or IL-10R1 S138G homozygous mutation.

The numbered statements below are also contemplated by the invention:

18. A method for detecting the presence of drug-resistant chronic lymphocytic leukemia in a mammal, comprising identifying in a nucleic acid sample from the mammal nucleotide 552 of SEQ ID NO:2, wherein the presence of a guanine (G) nucleotide at nucleotide 552 or the deletion of an adenosine (A) at nucleotide 552 in both alleles is indicative of the mammal being having drug-resistant chronic lymphocytic leukemia.

19. The method of statement 18, further comprising contacting the sample with at least one oligonucleotide probe to form a hybridized nucleic acid and amplifying the hybridized nucleic acid.

20. The method of statement 19, wherein IL10RA or a portion thereof is amplified.

21. The method of statement 19 or 20, wherein the amplification of the hybridized nucleic acid is carried out by polymerase chain reaction, strand displacement amplification, ligase chain reaction, or nucleic acid sequence-based amplification.

22. The method according to any one of statements 19 to 21, wherein at least one oligonucleotide probe is immobilized on a solid surface.

23. A method for detecting the presence of a biomarker associated with drug-resistant B cell malignancy, comprising determining the presence of the biomarker in a physiological sample from a mammal, wherein the sample comprises nucleic acid.

24. The method of statement 23, further comprising contacting the sample with at least one oligonucleotide probe to form a hybridized nucleic acid and amplifying the hybridized nucleic acid.

25. The method of statement 24, wherein IL10RA or a portion thereof is amplified.

26. The method of statement 25, wherein the amplification of the hybridized DNA is carried out by polymerase chain reaction, strand displacement amplification, ligase chain reaction, or nucleic acid sequence-based amplification.

27. The method according to any one of statements 24 to 26, wherein at least one oligonucleotide probe is immobilized on a solid surface.

28. The method of any one of statements 23 to 27, wherein the biomarker comprises an IL10RA gene having an G at nucleotide 552 of SEQ ID NO:2 or the deletion of an adenosine (A) at nucleotide 552 of SEQ ID NO:2.

29. The method of any one of statements 23 to 27, wherein the IL10RA gene encodes a protein having a G at amino acid residue 159 of SEQ ID NO:1.

30. The method of any one of statements 23 to 29, wherein the drug resistance is CpG-B ODN drug resistance.

31. The method of any one of statements 23 to 30, wherein the B cell malignancy is drug-resistant chronic lymphocytic leukemia (CLL).

32. A method for diagnosing drug-resistant B cell malignancy in a mammal comprising detecting the presence of a biomarker in a physiological sample from the mammal the sample, wherein the presence of the biomarker is indicative of drug-resistant B cell malignancy.

33. The method of statement 32, wherein the sample comprises nucleic acid.

34. The method of statement 32 or 33, further comprising contacting the sample with at least one oligonucleotide probe to form a hybridized nucleic acid and amplifying the hybridized nucleic acid.

35. The method of statement 34, wherein IL10RA or a portion thereof is amplified.

36. The method of statement 35, wherein the amplification of the hybridized DNA is carried out by polymerase chain reaction, strand displacement amplification, ligase chain reaction, or nucleic acid sequence-based amplification.

37. The method of any one of statements 32 to 36, wherein the biomarker comprises an IL10RA gene has an G at nucleotide 552 of SEQ ID NO:2 or a deletion of an adenosine (A) at nucleotide 552 of SEQ ID NO:2.

38. The method of any one of statements 32 to 36, wherein the IL10RA gene encodes a protein that has a G at amino acid residue 159 of SEQ ID NO:1.

39. The method of statement 32, wherein the biomarker is a IL-10R1 protein.

40. The method of statement 39, wherein the IL-10R1 protein has a G at amino acid residue 159 of SEQ ID NO:1.

41. The method of any one of statement 32 to 40, wherein the drug resistance is CpG-B ODN drug resistance.

42. The method of any one of statements 32 to 41, wherein the B cell malignancy is drug-resistant chronic lymphocytic leukemia (CLL).

43. A method of treating a human B cell malignancy in a patient with an IL10RA deletion or mutation comprising administering an effective dose of IL-10 for an effective period of time to the patient in need thereof.

44. The method of statement 43, wherein the B cell malignancy is drug-resistant chronic lymphocytic leukemia (CLL).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for detecting the presence of a biomarker in a mammal, comprising identifying in a nucleic acid sample from the mammal a guanine (G) at nucleotide 552 of SEQ ID NO:2 or a deletion of an adenosine (A) at nucleotide 552 of SEQ ID NO:2.

2. (canceled)

3. (canceled)

4. The method of claim 1, wherein the method comprises identifying in a nucleic acid sample from the mammal a deletion of an adenosine (A) at nucleotide 552 of SEQ ID NO:2.

5. (canceled)

6. A method for detecting the presence of a biomarker in a mammal, comprising identifying in an amino acid sample from the mammal a glycine (G) at residue 159 of SEQ ID NO:1 or a deletion of a serine (S) at residue 159 of SEQ ID NO:1.

7. (canceled)

8. The method of claim 6, wherein the method comprises identifying in an amino acid sample from the mammal a glycine (G) at residue 159 of SEQ ID NO:1.

9. The method of claim 6, wherein the method comprises identifying in an amino acid sample from the mammal a deletion of serine (S) at residue 159 of SEQ ID NO:1.

10. (canceled)

11. A method for detecting the presence of drug-resistant B cell malignancy in a mammal, comprising identifying a biomarker in a nucleic acid sample from the mammal nucleotide 552 of SEQ ID NO:2, wherein the presence of a guanine (G) at nucleotide 552 or the deletion of an adenosine (A) at nucleotide 552 in both alleles is indicative of the mammal having drug-resistant B cell malignancy.

12. The method of claim 11, further comprising contacting the sample with at least one oligonucleotide probe to form a hybridized nucleic acid and amplifying the hybridized nucleic acid.

13. The method of claim 12, wherein IL10RA or a portion thereof is amplified.

14. The method of claim 12, wherein the amplification of the hybridized nucleic acid is carried out by polymerase chain reaction, strand displacement amplification, ligase chain reaction, or nucleic acid sequence-based amplification.

15. The method according to claim 12, wherein at least one oligonucleotide probe is immobilized on a solid surface.

16. The method of claim 11, wherein the drug resistance is CpG ODN drug resistance.

17. The method of claim 11, wherein the drug resistance is CpG-B ODN drug resistance.

18-42. (canceled)

43. A method of treating a human B cell malignancy in a patient with an IL10RA deletion or mutation comprising administering an effective dose of IL-10 for an effective period of time to the patient in need thereof.

44. The method of claim 43, wherein the B cell malignancy is drug-resistant chronic lymphocytic leukemia (CLL).


SEQUENCE LISTING (2 sequences)

SEQ ID NO: 1 - protein, 578 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 2 - DNA, 3672 bp, Homo sapiens
gtcagtcccagcccaagggtagctggaggcgcgcaggccggctccgctccggccccggacgatgcggcgcgcccaggatgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacagggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagtgactcgggctgagaggctgcttttgattttagccatgcctgctcctctgcctggaccaggaggagggcccctggggcagaagttaggcacgaggcagtctgggcacttttctgcaagtccactggggctggccccagccaggccctgcagggctggtcagggtgtctggggcaggaggaggccaactcactgaactagtgcagggtatgtgggtggcactgacctgttctgttgactggggccctgcagactctggcagagctgagaagggcagggaccttctccctcctaggaactctttcctgtatcataaaggattatttgctcaggggaaccatggggctttctggagttgtggtgaggccaccaggctgaagtcagctcagacccagacctccctgcttaggccactcgagcatcagagcttccagcaggaggaagggctgtaggaatggaagcttcagggccttgctgctggggtcatttttaggggaaaaaggaggatatgatggtcacatggggaacctcccctcatcgggcctctggggcaggaagcttgtcactggaagatctcaaggtatatattttctggacactcaaacacatcataatggattcactgaggggagacaaagggagccgagaccctggatggggcttccagctcagaacccatccctctggtgggtacctctggcacccatctgcaaatatctccctctctccaacaaatggagtagcatccccctggggcacttgctgaggccaagcactcacatcctcactttgctgccccaccatcttgctgacaacttccagagaagccatggttttttgtattggtcataactcagccctttgggcggcctctgggcttgggcaccagctcatgccagccccagagggtcagggttggaggcctgtgcttgtgtttgctgctaatgtccagctacagacccagaggataagccactgggcactgggctggggtccctgccttgttggtgttcagctgtgtgattttggactagccacttgtcagagggcctcaatctcccatctgtgaaataaggactccacctttaggggaccctccatgtttgctgggtattagccaagctggtcctgggagaatgcagatactgtccgtggactaccaagctggcttgtttcttatgccagaggctaacagatccaatgggagtccatggtgtcatgccaagacagtatcagacacagccccagaagggggcattatgggccctgcctccccataggccatttggactctgccttcaaacaaaggcagttcagtccacaggcatggaagctgtgaggggacaggcctgtgcgtgccatccagagtcatctcagccctgcctttctctggagcattctgaaaacagatattctggcccagggaatccagccatgacccccacccctctgccaaagtactcttaggtgccagtctggtaactgaactccctctggaggcaggcttgagggaggattcctcagggttcccttgaaagctttatttatttattttgttcatttatttattggagaggcagcattgcacagtgaaagaattctggatatctcaggagccccgaaattctagctctgactttgctgtttccagtggtatgaccttggagaagtcacttatcctcttggagcctcagtttcctcatctgcagaataatgactgacttgtctaattcgtagggatgtgaggttctgctgaggaaatgggtatgaatgtgccttgaacacaaagctctgtcaataagtgatacatgttttttattccaataaattgtcaagaccacaggaaaaaaaaaaaaaaaaaaa